SEQUENCE LISTING (48 sequences)

SEQ ID NO: 1 - DNA, 621 bp, Homo sapiens  1887    OCA2-rs2311470
  misc_feature (426)..(426): n is g or c
agcaaatataacatgtggatgagactaactttgaaatttacgttcagaaaatattgatctcacattcatgatttttaaccctcaggggaaaagaaagtatttgactttggttcagtgagtttacagcactgtgatcaatacagagattaacctttgaaaaggcacccaatattacattagcaactgttttgacctttgagaaaatatgaatgtctgcctggctacccttgaagcagcagacagaatgagggctttataaatctacaaagaattttattatgtttactgttagtcaatggtaatgatccctttgaatcacactgaacaaagccaggtcattttcaaatattgcttcgaatacagtattttctagatggtttattctctagatatttttttcactttttcatttaatttattaacttnatgtttttaaagcctcacaataattagctatgttctaagaaaccacaaaagaaaacaaaatgaggtgacaaaagctttactggaagctaaattccacaataaaattcaacttaatgtgtctctatgtttaaagaaatagtataaatcagcattatcaattagtgagacactctttaattgactggtgtatcttac

SEQ ID NO: 2 - DNA, 571 bp, Homo sapiens  1867    OCA2-rs1375170
  misc_feature (497)..(497): n is g or t
gcagggtcagggaatggtacagccctacaaatatgccattagaaggcagtaactaaaactttcaggacactcaagggttactgtgcttagaggctggtaagtttcaccaaatatgaagacctctaatgagaacacacagacacagacacagaggcaggatttttgtccttgataaacacaagtaccagttacccaggctgtcccctgaatgccagctcattcacattctaaagcactcacattgtcagagaaagacagtggcaggctgagctcccgggatggactgagagacagctgcatcgatgcacatgccctgagcacactcaagtatggtctctgtggccttgggagtggcggactggactgagctctgccccgaggactgcacaggctcatggagaatacagctcacatacaactgccgtgatggacctgtgacggctatgctagcctcacacaccaaggtatcataggggtctctcagctaaatccatacncttgctgaggccatgggggaaccaaccagagcttaactgaacattggagctcttctgtggtctcaagcatcttt

SEQ ID NO: 3 - DNA, 308 bp, Homo sapiens  1905    OCA2-rs1004611
  misc_feature (68)..(68): n is c or t
acaaatttcttacagaaacttcacagccactgtgtttcattgattacaataatgcaaagtgcatcaangtcagaaaggtgcaccagaggctggcacggctgactagagaatggtgcttcctggctgtagaatctggtacctgatgtcatgctgtgaatgttagcaggctcagggtgcctggcagggagcagccgtgataagtggcttagcacaggggacccagcatgcagtttagggtcaggggactgtgtgtgagttccggctggattgctttcaagatctgtgctcctgtgcctctctgcatcctg

SEQ ID NO: 4 - DNA, 227 bp, Homo sapiens  1869    OCA2-rs1874835
  misc_feature (171)..(171): n is g or t
caggttgagagatctgcatcacaattggatcttaagaaagccaaacaatgtttttcattaaacaagagagctcttaataagtagcttttaaaaacatgattattctcattattcaacagaatggaagccactttctttgcaatcgtcaggcaaactccagaggaacagggnagacgatcgcttctgcatttgcaaagatggaccaagctgccccacggacattatcc

SEQ ID NO: 5 - DNA, 622 bp, Homo sapiens  1908    OCA2-rs895829
  misc_feature (533)..(533): n is c or t
ctgtgttctaattacagtacagccacagataaaaataatcacgccttctgctgagaggccgttcatgacccttccaaacttggaatcatgcagggatcaacctcagtctagctctgagcattgctggcctcagagctaatcaacccaaatgcaaatccacaagttaaaaagggaaatcagtagaagctggtggggaaacatgagaataaatccataaacccccaaaataattccaccccatcagatactctataaactgtgaatagatgcaaagcaaataatgacactacgatacgatgtaaaactagaatactatttctgcattttcacagaggtctaatgccctgcattaagcaagctcacaggcttcgacaaaattccaaattgagaaatgttaaattttgcaattctgtattttatcagaatgttaccaataattcctcaatgggtactggcccaggtgctgttctaggaagggggtaatggacaggtgagttgctggcctatccccttactcaggcacgtaagactcngtgagcaagtataacgcaaggtgaagaccgtagaagcagacagggatggcgatggcaatgactgcgctgccaggtaaggcttcacagag

SEQ ID NO: 6 - DNA, 596 bp, Homo sapiens  1916    OCA2-rs1498519
  misc_feature (369)..(369): n is a or c
attttcacttggcgccctccacctagcaacctctatttggcccagaaccaagggcctggatccccagggtagcctgcctgtgttcaagggatggctgggaggtaggtgccctcctgggaagcagggctgctgggctgggaggagactgtgcctcagggaaaaggcgaggctgccttggtactgggtaggagtgagtggtgaggacctagccaaaggtaggtgagaggacaccctctgtgtccatttaacaaagcagagtttcagacagggtgtgaggcttccagagagacatttaacattcactgagcttcaggagagctgaccaccccaagccaaggccaacactgcctagtgtcctcactaagcaanccctcaggacacaggttccaactcagccactgagggagagagagacaaggcggtgtcagtgtccttcatagagaaggagcgaatgtctgggttggctgctcccagattccttagctgggaactctgagcacacattcttttcagagatgaccatagggtccttctcagggcatgcttcagtgccatgagtagaacatccatactggatttttttatttaaaaaatgt

SEQ ID NO: 7 - DNA, 622 bp, Homo sapiens  1879    OCA2-rs895828
  misc_feature (509)..(509): n is g or c
ctgtgttctaattacagtacagccacagataaaaataatcacgccttctgctgagaggccgttcatgacccttccaaacttggaatcatgcagggatcaacctcagtctagctctgagcattgctggcctcagagctaatcaacccaaatgcaaatccacaagttaaaaagggaaatcagtagaagctggtggggaaacatgagaataaatccataaacccccaaaataattccaccccatcagatactctataaactgtgaatagatgcaaagcaaataatgacactacgatacgatgtaaaactagaatactatttctgcattttcacagaggtctaatgccctgcattaagcaagctcacaggcttcgacaaaattccaaattgagaaatgttaaattttgcaattctgtattttatcagaatgttaccaataattcctcaatgggtactggcccaggtgctgttctaggaagggggtaatggacaggtgagttgctggcctatcnccttactcaggcacgtaagactccgtgagcaagtataacgcaaggtgaagaccgtagaagcagacagggatggcgatggcaatgactgcgctgccaggtaaggcttcacagag

SEQ ID NO: 8 - DNA, 263 bp, Homo sapiens  1991    TYRP1E6_354_886938
  misc_feature (172)..(172): n is c or t
ccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctggtaagacattttcatatgcnttttgcatgctcagctgggcrgattgtttagatggcatagttatcagttcaagctgagcactcagcgcataaaaacactttcaaaataagg

SEQ ID NO: 9 - DNA, 321 bp, Homo sapiens  1877    TYRP1-rs683
  misc_feature (181)..(181): n is g or t
tagttcacttatatcttcacatactcaacctttaaatcatgccaaagatcttcacactatccttaaatatcatggagtttagtgtcattcaccattttaaatagaactgaaaattaaaacccattgaaaagattctgaaagggtcttcccagctttgaaaagtatgcctagaactttaatnctaacatatgcttgtattagaaagaaggtaataaagtgaaagaaaatacagttaataactcaatctattatattcaaccaggtggttttgtgatactaatgcataagagagtagggcatttgttagaccacagactgatt

SEQ ID NO: 10 - DNA, 785 bp, Homo sapiens  1948    TYRP_3_217485
  misc_feature (360)..(360): n is a or c
tttgtctttttatttttatcttcctttccaaataggtcgggagtttagtgtacctgagataattgccatagcagtagttggcgctttgttactggttgcactcatttttgggactgcttcttatctgattcgtgccagacgcagtatggatgaagctaaccagcctctcctcactgatcagtatcaatgctatgctgatatgaaaaactccagaatcctaatcagtctgtggtctaacaaatgccctactctcttatgcattagtatcacaaaaccacctggttgaatataatagattgagttattaactgtattttctttcactttattaccttctttctaatacaagcatatgttagnattaaagttctaggcatacttttcaaagctgggaagaccctttcagaatcttttcaatgggttttaattttcagttctatttaaaatggtgaatgacactaaactccatgatatttaaggatagtgtgaagatctttggcatgatttaaaggttgagtatgtgaagatataagtaagtgaactaccatgctttgtttacgtgtaaaggaaaataatgtttgatagtaaatgtccacttaaaatacatgaatgggcatttctaaaatgttaaaacataaacwcatttccattcatggatatttgtcaacagatttaaagaaaaccacagttattaattaaagaaaattaattatgtgtagttataaaccaatgaaattttgattaaccttttcaaattaatgttccagtttgaagaccaatcaaat

SEQ ID NO: 11 - DNA, 543 bp, Homo sapiens  RS724657
  misc_feature (32)..(32): n is a, c, g, or t
actggtaaggcattgcgagaaatcacacaggngaaataaagcttgccaataaggggaactcattaaatcagctaccacagtggatgacaacagcttaatcacgtgggagaaattctggtaaatggtgcaaaactgtgcgtgatgtactgaaagatcagagggatagggcaaggcacngacagagtctgctatgattgtaacaggaggaggtagaaattaaacaggaatggtgactccatataatttggcttcctatttctgtgactctacacactgaagatatcaatcaaatcatagagtgctattttaaatagaaggaagctcaattttcaatcacatataactgaaaccatagtataactttttaagtattttaattcgtgctgtttggggtattcaaatatatcctgaatttaaactatagtaaccctgaacccagtattcatttgcaaaaactatcaacatctacatgattaaatactattttccagcanacattccaaaaagttgcttctgtgaacagtatgaatagaaaagtctaga

SEQ ID NO: 12 - DNA, 496 bp, Homo sapiens  RS1373904
  misc_feature (344)..(344): n is c or t
ctatattgggaatcgtttggacatctagaatttgatggggcaaaaatgtcaagtttcctgtgggtttaagggagcataggtaggctacaacggtctttgtaaaataggaagaggctatgtagagtgtaccatcaaagctggatgggtgcggtaaatggtccgctggagagcactttgtttgaatgggtgtgtggtagtggcgaggaggcgagttgcagaggtggccccctaacacaacctatttctcatcctccctcgctatcaacaccctggcattagtaggtgaaaaagatgatcacagttgggaaaatagagaacaaatcatgctttatgccccagtgctngtctggtctaagctggggaggtgagaagctctaaatttaaatcaggttagacttaataatagtctaaagtttaccacaggtgaaacaaatgtctaaagatgggtaaagaaacatggctgaatgctttaaaacagatctccactgcaagcatt

SEQ ID NO: 13 - DNA, 461 bp, Homo sapiens  RS440383
  misc_feature (24)..(24): n is c or t
tattgtattataaacccagatganatcttagctgggaatgtggctttctttcctacctcaggcatagctatgcctgtgtaataatctcttacactggcagagagtactattgatctgtcaatcaactggcatgttcaacctccagatgtgggctcacttgcaaaatgcaattctgggagtagctgtttgagttattgtaactgggactgatctgggcctcttttgaagggtatgctgcagtcgatagcaattttcattgtcttggtgtatggggtgtgagtgtctggataataggtctgcaggatagtaaaaataatgattttttcttgattataagttacctttgaaatacatatgattgaggccattttgtactatcttttgtgccctacttggaggacatcagcctcttcacattcttctccccactacaagtatttctaggacttgcatgggacatt

SEQ ID NO: 14 - DNA, 291 bp, Homo sapiens  RS4128358
  misc_feature (137)..(137): n is g or c
ctgtggaagatgcgtgtgggattgtgagaacacacctgggtgcagagagcctggttagaaagtgaatttaatagtgcaggcaacagaggatggaagtgtgaactaaggcaggagcagagcagttggcttcaagacanatttaagagataagacggaaataatgtggtgaccaagagggtatgagtagacgagaaactgacaatatctatgggtatctcctccatcttgccatcataccaaattactttttatttctatgtcactgtcttcatttgcacactgtcactcact

SEQ ID NO: 15 - DNA, 215 bp, Homo sapiens  RS2366267
  misc_feature (169)..(169): n is a or g
taaatgtgaaaaatgaaaaccaagatcagaaaatagttagaaaattataaactgtgggcgggtgttccagtgtgcttaccagagcactcactcgccggtgggcagggaaatgccgtgggaggcagcagcacagattcttctgtcttttatccacagcaaaaatacccancagtgacaacatccagtgctgatgggcaggcaggaggaccaggtca

SEQ ID NO: 16 - DNA, 591 bp, Homo sapiens  RS1430104
  misc_feature (318)..(318): n is a or g
attgactttcaatcactttgtgagagtgaacactgttactgcagggtctagctgctccacccattgttctagatgggagataaaacagcctaactctagctggatgctcagtccaaactctaggatgagggtcaaggcagggactgcactattcattcatgtttgcttttctgggactcctataactgatgccttggatttgtgggatggttggttatgaccagtggctacacctgaggaactcactgtagcttgatggttcatatagatttgagattaccaaaatgttattcaacattcatactagatggtcttgantatacttcaaattcagcatgttctcatctgaactcacatctcaacatcaccacactgtccctttcctttttcttatattttcagagtatcagtataaatctcattgcaccaggcagaatctgaggagaaatccttgcagctccttcttcctgattcatcatcaaagcctactgattttgcctccaaaccattcacttctgttcctctttacatatggacaatgggaggaactgactaaccacttgggcttgtcattctgtttgacccctttcc

SEQ ID NO: 17 - DNA, 376 bp, Homo sapiens  RS717389
  misc_feature (122)..(122): n is c or t
tagtgtcatcctaaggttggtgtcaattagggcaatggtcgtccttcttctcatctagtaagacagacgaagatacgatggggtctaagttcttcttgtcagtttccttggccaggcttgancataggcccacgctgaactggtaattgtggcagtgggaaagacatgcacttcttggtttagagtccagaggtttccatcttacgctgagaatgggttacacatgtagagatattgatcccttcaggccttagggctgccattttttcttgccccaaagccactgtcgcatctcgccagtcatctccaagcagagtggcctcttccttttatcctacaggctatacaagcactgttttttttttttaattcgtca

SEQ ID NO: 18 - DNA, 50 bp, Homo sapiens  RS2369860
  misc_feature (26)..(26): n is a or g
gtggttttaaacaaacagatcttgcnagcactcactcgctattgtgagga

SEQ ID NO: 19 - DNA, 537 bp, Homo sapiens  RS1209180
  misc_feature (220)..(220): n is a or g
gcatgtccctattggggaaatctatttatgttttcaattggggatatttttaaagttttttaaaaagatcttggatctaacttctgtttcagcggaaacggcactattggagattggatagcaaatgtattaccctctttcagaatgacacaggaagcaggtactacaaggtaagagtgggctctttctttgtagtttcatacctcttattgctttttcntgggaatgtagagtataagaaataggaaatacatccaacaaacgtggctatttttgtgcttcctccaggaactattttttttttcttctctatagtctacaaatttcttgggattcaggttgggacctcttgattcttctgtgaattttaaatttccctggggagtgtttctggtaccatgggatgatgggagagggagggaggtgaagattaattgaatatgttagcagagaagtgtgatttaactagaaaattaatggtcacagcagatcagctcaaagtaaactcctgatatccactgcatctagcacactgct

SEQ ID NO: 20 - DNA, 257 bp, Homo sapiens  RS1073801
  misc_feature (178)..(178): n is a or c
ggctccatgtatattagttcatcatattctcagaagcacccctgtgaaccctgtactgttagtagccccattttgtggagggagagactaaaccacagagagtccatcccaggacctgggaggaacaaagctgggatttaaggccaggcagcttgggaccctaagccaatgtgtgtantccttgtgacctactgcctttctgctaagctcctggggccccatcattgacctgcagacagacacaagcacctcttaaa

SEQ ID NO: 21 - DNA, 51 bp, Homo sapiens  RS1112619
  misc_feature (26)..(26): n is c or t
gcttgcagtgatggacacccaaatangctggcttgatcattacacagtcta

SEQ ID NO: 22 - DNA, 546 bp, Homo sapiens  RS1998463
  misc_feature (402)..(402): n is g or c
gaaacttgtgggggaagttctagattaaaatcccaacacctttggtattggtgatttgaatgtcacttgctggaaccagctagtgacctctgttctatcctgtctttggctcaattttaatcacaaggaacaatcagactatagagaggtgtgtgctttttctgtagtcaaataagactggctttcattctaggaagaattctccctggagctccgtttaacctgtgttttttatcaggatgctatgaagacctattaggacctaggatacaacaaattgagcataatcacctcaggctctatggcttatagcaaagatatacctccttgttagcctgttgggaagaataaatgtttttggtgtttaaaagaaaaaaaaaactattaatgttgcataataanccacgaagacaaaatgagtagaatatctccaatcttctatacccatgttctttgcctgaattttattgactttttactttcaagattataattgaagttcaatttatcttgctacctaattgttccagtacaaaagaattaaat

SEQ ID NO: 23 - DNA, 449 bp, Homo sapiens  RS950303
  misc_feature (107)..(131): n is a, c, g, or t
tcaggggaccagtaccagagttagaatggacaaagagtagcatgtgattctaataatgaaagaccaaggaaaggattgtggccaccagcggtggttgagagattaannnnnnnnnnnnnnnnnnnnnnnnntcatcagtgtgcacnttatcccactccaccccatctcaccagagttgtgaggctacccagggagaagggagaagaaaggggaagaaagaaattgcaaaagattatttccttaaaattgattagaaaaaaattcatctaatccagataagtatctttgagtgaaaaataactatatgttgtctgcttcaaaattttactgtgtaaatattaacatattgaatattgggtgctccccacccaagcctttccctcctccccatgcatccttcagtttgaagcatctttaagcactggtggaagtgcaggtctaagtctaga

SEQ ID NO: 24 - DNA, 310 bp, Homo sapiens  RS1887027
  misc_feature (207)..(207): n is a or g
atttcagagagcactcgatgctcccaaaagaagaagttgacagagcccacgtcccctcagcccttcacacagcctccccttgtcttcctctcctactcagcactgtggctgcactaggacccctcatgatgaccaagatcctcataaagaggacaagaagttcatccagtaccagtcaggccaatatcccaaaattagcataactgncagaaatgcagaggcatcaacaactcctcttgacaacagggttggtagaagaatcaagggcctaattttgatgcctggcacaggttatgggagcagcagagta

SEQ ID NO: 25 - DNA, 431 bp, Homo sapiens  RS726865
  misc_feature (337)..(337): n is c or t
agggcaatgatgagctagcctgtaattatcagtgcctatatcctctctacagcttgtagggacatatattattatgttaaaaaaaaaaaagcaatatgaggcttgttagtgcttggaagtatacaagacatacttccaagttacaaagacatacttccaagttacaaagacatacttccaagtatgaacaaaacaaacacatgcgtcccttgtggcagagcagagctggcccttccccagatgacacacctcagggacttcaccttataccttgcagtgttctgtatttggttttccatgtgaggcagcaacgggttcaaagagagaccatctgagnacaagtgatgggcgtggcacatccagggagatcaagaaccaggccagatgcagagacctgacctgtgagccaagtctgggcacctgaatctaga

SEQ ID NO: 26 - DNA, 41 bp, Homo sapiens
  misc_feature (21)..(21): n is c or t
tatctaaccctcactgagctntgcagggggtacacagccga

SEQ ID NO: 27 - DNA, 41 bp, Homo sapiens
  misc_feature (21)..(21): n is c or t
cacatgttcattgggatttgncttgttctcctggtctgctt

SEQ ID NO: 28 - DNA, 41 bp, Homo sapiens
  misc_feature (21)..(21): n is a or g
cttgtcttcttcttttccccntagatgatcttagtagccat

SEQ ID NO: 29 - DNA, 41 bp, Homo sapiens
  misc_feature (21)..(21): n is a or g
gtgcatgaaaaggtgggggcngttgagcccacagctcactg

SEQ ID NO: 30 - DNA, 41 bp, Homo sapiens
  misc_feature (21)..(21): n is a or c
gtgaaataatttccatgattncttcctaaatattgaatata

SEQ ID NO: 31 - DNA, 41 bp, Homo sapiens
  misc_feature (21)..(21): n is g or t
cagtgtgaactgtgtaggttntgtgtggtcccctgtgcctg

SEQ ID NO: 32 - DNA, 41 bp, Homo sapiens
  misc_feature (21)..(21): n is g or t
atgaaaaaaaagttgttgttntttttttttttcctattttt

SEQ ID NO: 33 - DNA, 41 bp, Homo sapiens
  misc_feature (21)..(21): n is g or t
atgtctgtgtgtgtgcaccantgtgaactgtgtaggttgtg

SEQ ID NO: 34 - DNA, 41 bp, Homo sapiens
  misc_feature (21)..(21): n is a or g
tcctcccccgtctcttttgcnttcagcgtgacccatcttcc

SEQ ID NO: 35 - DNA, 41 bp, Homo sapiens
  misc_feature (21)..(21): n is a or g
ccaggcataccggctctcccngggacgggtgtgggccatga

SEQ ID NO: 36 - DNA, 41 bp, Homo sapiens
  misc_feature (21)..(21): n is t or c
gtcagtgaaacatccccaaanggtaagcttcagcattgtcc

SEQ ID NO: 37 - DNA, 51 bp, Homo sapiens
  misc_feature (26)..(26): n is g or c
catgtttctccctttaagcaaaaggnggctctcttttgtttccttttgcaa

SEQ ID NO: 38 - DNA, 41 bp, Homo sapiens
  misc_feature (21)..(21): n is a or c
gatgttatatgttttgaaagnagaaatacttcaattagtaa

SEQ ID NO: 39 - DNA, 41 bp, Homo sapiens
  misc_feature (21)..(21): n is a or g
gtggcccacagtgagtgcttnggacatctggatgcagctat

SEQ ID NO: 40 - DNA, 41 bp, Homo sapiens
  misc_feature (21)..(21): n is a or g
atcgtgcacagaactctggcngccatgctgggttcccttgc

SEQ ID NO: 41 - DNA, 41 bp, Homo sapiens
  misc_feature (21)..(21): n is a or t
taaaatgttaaaacataaacncatttccattcatggatatt

SEQ ID NO: 42 - DNA, 41 bp, Homo sapiens
  misc_feature (21)..(21): n is a or g
cactgggatctgcctgtgacnctcttgtggggcctggtccc

SEQ ID NO: 43 - DNA, 41 bp, Homo sapiens
  misc_feature (21)..(21): n is a or g
gaagttaaaacattgttagtnaattctgggtgaatggatgg

SEQ ID NO: 44 - DNA, 41 bp, Homo sapiens
  misc_feature (21)..(21): n is c or g
gaggttggatgttggggcttntgcatcaactccgtgttttc

SEQ ID NO: 45 - DNA, 41 bp, Homo sapiens
  misc_feature (21)..(21): n is c or t
atatctctgaatcctaatganagcagaaacatgttaatgag

SEQ ID NO: 46 - DNA, 41 bp, Homo sapiens
  misc_feature (21)..(21): n is a or g
ctcaagaactctgataaacantatgtttggcattctgttcc

SEQ ID NO: 47 - DNA, 41 bp, Homo sapiens
  misc_feature (21)..(21): n is c or t
tcatgggttttgtgagtgtangcatgctttagaagagctct

SEQ ID NO: 48 - DNA, 41 bp, Homo sapiens
  misc_feature (21)..(21): n is c or t
tagactagaaatgagcccagntgaacactctatatgaaaaa

(19) United States
(12) Patent Application Publication
(10) Pub. No.: US 2008/0193922 A1
(43) Pub. Date: Aug. 14, 2008

(54) Methods and Compositions for Inferring Eye Color and Hair Color

(76) Inventor: Tony N. Frudakis, Bradenton, FL (US)

Correspondence Address: DLA PIPER US LLP, 4365 EXECUTIVE DRIVE, SUITE 1100, SAN DIEGO, CA, 92121-2133, US

(21) Appl. No.: 10/589,291

(22) PCT Filed: Feb. 11, 2005
(86) PCT No.: PCT/US2005/004513

§ 371 Date: Jun. 4, 2007

Related U.S. Application Data
(60) Provisional application No. 60/544,788, filed on Feb. 13, 2004.
(60) Provisional application No. 60/548,370, filed on Feb. 27, 2004.

(51) Int. Cl. C12Q 1/68

(52) U.S. Cl. 435/6

(57) ABSTRACT

Methods for inferring eye color or eye shade of an individual from a nucleic acid sample of the individual by detecting the nucleotide occurrence of an eye color related single nucleotide polymorphism (SNP) as set forth in SEQ ID NOS: 1 to 7 and, optionally, SEQ NOS:8 to 10 and/or SEQ ID NOS:26 to 48, are provided. Also provided are methods for inferring hair color or hair shade of an individual from a nucleic acid sample of the individual by detecting the nucleotide occurrence of a hair color related SNP as set forth in SEQ ID NOS: 11 to 25. Methods for inferring eye color/shade and/or hair color/shade of an individual from a protein sample of the individual by detecting an amino acid residue encoded by the nucleotide occurrence of an eye color related SNP or a hair color related SNP, respectively, also are provided. In addition, compositions, including oligonucleotides and antibodies, useful for practicing such methods are provided, as are kits for performing the methods.

This application claims the benefit of priority under 35 U.S.C. § 119 of U.S. Ser. No. 60/548,370, filed Feb. 27, 2004, and U.S. Ser. No. 60/544,788, filed Feb. 13, 2004, the entire content of each of which is incorporated herein by reference.

### BACKGROUND OF THE INVENTION

1. Field of the Invention

The invention relates generally to methods of determining pigmentation traits of an individual, and more specifically to methods of inferring eye color or hair color of an individual by identifying single nucleotide polymorphisms (SNPs) associated with eye color or hair color, respectively, in a nucleic acid sample of the individual, and to compositions useful for practicing such methods.

2. Background Information

Biotechnology has revolutionized the field of forensics. More specifically, the identification of polymorphic regions in human genomic DNA has provided a means to distinguish individuals based on the occurrence of a particular nucleotide at each of several positions in the genomic DNA that are known to contain polymorphisms. As such, analysis of DNA from an individual allows a genetic fingerprint or “bar code” to be constructed that, with the possible exception of identical twins, essentially is unique to one particular individual in the entire human population.

In combination with DNA amplification methods, which allow a large amount of DNA to be prepared from a sample as small as a spot of blood or semen or a hair follicle, DNA analysis has become a routine tool in criminal cases as evidence that can free or, in some cases, convict a suspect. Indeed, criminal courts, which do not yet allow the results of a lie detector test into evidence, admit DNA evidence into trial. In addition, DNA extracted from evidence that, in some cases, has been preserved for years after the crime was committed, has resulted in the convictions of many people being overturned.

Although DNA fingerprint analysis has greatly advanced the field of forensics, and has resulted in freedom of people, who, in some cases, were erroneously imprisoned for years, current DNA analysis methods are limited. In particular, DNA fingerprinting analysis only provides confirmatory evidence that a particular person is, or is not, the person from which the sample was derived. For example, while DNA in a semen sample can be used to obtain a specific “bar code”, it provides no information about the person that left the sample. Instead, the bar code can only be compared to the bar code of a suspect in the crime. If the bar codes match, then it can reasonably be concluded that the person likely is the source of the semen. However, if there is not a match, the investigation must continue.

An effort has begun to accumulate a database of bar codes, particularly of convicted criminals. Such a database allows prospective use of a bar code obtained from a biological sample left at a crime scene; i.e., the bar code of the sample can be compared, using computerized methods, to the bar codes in the database and, where the sample is that of a person whose bar code is in the database, a match can be obtained, thus identifying the person as the likely source of the sample from the crime scene. While the availability of such a database provides a significant advance in forensic analysis, the potential of DNA analysis is still limited by the requirement that the database must include information relating to the person who left the biological sample at the crime scene, and it likely will be a long time, if ever, that such a database will provide information of an entire population. Thus, there is a need for methods that can provide prospective information about a subject from a nucleic acid sample of the subject.

### SUMMARY OF THE INVENTION

The present invention provides methods of inferring the natural eye color of a human subject from a nucleic acid sample or a polypeptide sample of the subject, methods of inferring the natural hair color of a human subject from a nucleic acid sample or a polypeptide sample of the subject, and compositions for practicing such methods. The methods of the invention are based, in part, on the identification of single nucleotide polymorphisms (SNPs) that, alone or in combination, allow an inference to be drawn as to eye shade or eye color and as to hair color. As such, the methods can utilize the identification of haploid or diploid alleles of SNPs and or haplotypes. The compositions and methods of the invention are useful, for example, as forensic tools for obtaining information relating to physical characteristics of a potential crime victim or a perpetrator of a crime from a nucleic acid sample present at a crime scene, and as tools to assist in breeding domesticated animals, livestock, and the like to contain a pigmentation trait as desired.

In one embodiment, the invention relates to a method of inferring eye shade or eye color of a human individual by determining the nucleotide occurrence of at least one (e.g., 1, 2, 3, 4, 5, etc.) SNP as set forth in any of SEQ ID NOS:1 to 10 and 26 to 48. Such a method can be performed, for example, by determining the nucleotide occurrence of at least one SNP of an oculocutaneous albinism II (OCA2) gene as set forth in any of SEQ ID NOS:1 to 7, the nucleotide occurrence of at least one SNP of a tyrosinase-related protein (TYRP) gene as set forth in any of SEQ ID NOS:8 to 10, or a combination of SNPs as set forth in any of SEQ ID NOS:1 to 10; and can further include determining the nucleotide occurrence of a SNP as set forth in any of SEQ ID NOS:26 to 48. An inferred eye color, which can be quantitated as described in Example 1, can be a lighter eye shade (e.g., green irises or blue irises), or can be a darker eye shade (e.g., brown irises or hazel irises). In one aspect, the method comprises identifying at least two nucleotide occurrences of the SNP position, including, for example, diploid alleles corresponding to at least one SNP position. In another aspect, the method comprises identifying a haplotype and/or diploid alleles of a haplotype comprising at least two SNP positions, and including at least one SNP as set forth in any of SEQ ID NOS:1 to 7 and/or SEQ ID NOS:8 to 10 and/or SEQ ID NOS:26 to 48.

A method for inferring eye color (shade) of a human subject from a nucleic acid sample of the subject can be practiced by identifying in the nucleic acid sample at least one eye color related SNP of an OCA2 gene, wherein the SNP comprises nucleotide 426 of SEQ ID NO:1, wherein a G residue indicates an increased likelihood of a lighter eye shade; nucleotide 497 of SEQ ID NO:2, wherein a T residue indicates an increased likelihood of a darker eye shade; nucleotide 68 of SEQ ID NO:3, wherein a T residue indicates an increased likelihood of a darker eye shade; nucleotide 171 of SEQ ID NO:4, wherein a T residue indicates an increased likelihood of a darker eye shade; nucleotide 533 of SEQ ID NO:5, wherein a C residue indicates an increased likelihood of a darker eye shade; nucleotide 369 of SEQ ID NO:6, wherein a C residue indicates an increased likelihood of a darker eye shade; or nucleotide 509 of SEQ ID NO:7, wherein a C residue indicates an increased likelihood of a darker eye shade. Such a method can include, for example, identifying one, two, three or more eye color related SNPs, including 1, 2, 3, 4 or more of the exemplified OCA2 SNPs.

In another embodiment, the present invention relates to compositions useful for sampling a nucleic acid sample to determine a nucleotide occurrence of at least one SNP informative of eye color. Such compositions include, for example, oligonucleotide probes that selectively hybridize to a nucleic acid molecule as set forth in SEQ ID NOS:1 to 7, or, optionally, to a nucleic acid molecule as set forth in SEQ ID NOS:8 to 10 and/or SEQ ID NOS:26 to 48, including one or the other of a nucleotide occurrence (i.e., alternative alleles) of a SNP (e.g., a nucleic acid molecule containing either a “G” or an “C” residue at the SNP position of SEQ ID NO:1 (marker 1887); or oligonucleotide primers that selectively hybridize to a position upstream or downstream (or both) of the nucleotide position such that a primer extension reaction or a nucleic acid amplification reaction can generate a product including the SNP position. Where the nucleotide occurrence of a SNP position is in a gene coding sequence, and the alternative forms of the SNP result in a change in the encoded amino acid, the composition for detecting the nucleotide occurrence at the SNP position can be an antibody that specifically binds to a polypeptide containing one or the other amino acid residue, but not to both such polypeptides.

In still another embodiment, the invention relates to a method of inferring natural hair color (i.e., the hair color that is determined by the genetic make-up of the individual) of a human individual by determining the nucleotide occurrence of at least one SNP as set forth in any of SEQ ID NOS:11 to 25 (e.g., nucleotide 494 of SEQ ID NO:11, nucleotide 344 of SEQ ID NO:12, etc.; see Sequence Listing). In one aspect, the method comprises identifying at least two (e.g., 2, 3, 4, or more) nucleotide occurrences of the SNP position, including, for example, diploid alleles corresponding to at least one SNP position. In another aspect, the method comprises identifying a haplotype and/or diploid alleles of a haplotype comprising at least two SNP positions, and including at least one SNP as set forth in any of SEQ ID NOS:11 to 25. For example, a method for inferring hair color can be performed by identifying in the nucleic acid sample one or more hair color related SNPs comprising nucleotide 177 of SEQ ID NO:11; nucleotide 344 of SEQ ID NO:12; nucleotide 24 of SEQ ID NO:13; nucleotide 137 of SEQ ID NO:14; nucleotide 169 of SEQ ID NO:15; nucleotide 318 of SEQ ID NO:16; nucleotide 122 of SEQ ID NO:17, nucleotide 26 of SEQ ID NO:18; nucleotide 220 of SEQ ID NO:19; nucleotide 178 of SEQ ID NO:20; nucleotide 26 of SEQ ID NO:21; nucleotide 402 of SEQ ID NO:22; nucleotide 146 of SEQ ID NO:23; nucleotide 207 of SEQ ID NO:24; and/or nucleotide 337 of SEQ ID NO:25.

In another embodiment, the present invention relates to compositions useful for sampling a nucleic acid sample to determine a nucleotide occurrence of at least one SNP informative of hair color. Such compositions include, for example, oligonucleotide probes that selectively hybridize to a nucleic acid molecule as set forth in SEQ ID NOS:11 to 25, including one or the other of a nucleotide occurrence of a SNP; or oligonucleotide primers that selectively hybridize to a position upstream or downstream (or both) of the nucleotide position such that a primer extension reaction or a nucleic acid amplification reaction can generate a product including the SNP position. Where the nucleotide occurrence of a SNP position is in a gene coding sequence, and the alternative forms of the SNP result in a change in the encoded amino acid, the composition for detecting the nucleotide occurrence at the SNP position can be an antibody that specifically binds to a polypeptide containing one or the other amino acid residue, but not to both such polypeptides. Also provided are kits comprising such compositions, including, for example, a kit containing one or a plurality of oligonucleotide probes useful for sampling an alternative allele of one or more eye color related SNPs and/or hair color related SNPs; and/or one or more primers (or primer pairs) useful for sampling a SNP position; or a combination of such probes and primers (or primer pairs).

An inference as to eye color (or hair color), according to the present methods, can be made by comparing the nucleotide occurrences of one or more SNPs of the test individual (i.e., the subject providing the nucleic acid sample to be tested) with known nucleotide occurrences of the eye color (or hair color) related SNPs that are associated with a known eye color/shade (or hair color/shade) (e.g., a G at nucleotide 426 of SEQ ID NO:1, which is associated with a lighter eye shade—e.g., green or blue). For example, the known nucleotide occurrences of eye color related SNPs that are associated with known eye colors can be contained in a table or other list, and the nucleotide occurrences of the test individual can be compared to those in the table or list visually; or can be contained in a database, and the comparison can be made electronically, for example, using a computer. Further, each of the known nucleotide occurrences of eye color related SNPs associated with an eye color/shade can be further associated with a photograph of a person from whom the corresponding eye color and nucleotide occurrence(s) was determined, thus providing a means to further infer eye color/shade) of a test individual. In one aspect, the photograph is a digital photograph, which comprises digital information that can be contained in a database that can further contain a plurality of such digital information of digital photographs, each of which is associated with a known eye color (or hair color) corresponding to nucleotide occurrence(s) of eye color (or hair color) related SNP(s) of the persons in the photographs.

Accordingly, the invention provides an article of manufacture comprising a photograph, including a photograph of one or both eyes (or of the hair), of a person having a known natural eye color (or natural hair color) and, associated with the known natural eye color (or natural hair color), known nucleotide occurrence(s) of eye color (or hair color) related SNP(s). Also provided is a plurality of such photographs, which can include photographs of different persons with the same eye color or eye shade (or natural hair color or shade), different persons with different eye colors or eye shades (or natural hair color or shade), and combinations of such photographs. In one embodiment, the photograph is a digital photograph, which comprises digital information. As such, the digital information comprising the digital photograph, or the plurality of digital photographs, can be contained in a database. Ii one aspect, the digital information for one or a plurality of the articles (photographs) is contained in a database, which can be contained in any medium suitable for containing such a database, including, for example, computer hardware or software, a magnetic tape, or a computer disc such as floppy disc, CD, or DVD. As such, the database can be accessed through a computer, which can contain the database therein, can accept a medium containing the database, or can access the database through a wired or wireless network, e.g., an intranet or internet.

### BRIEF DESCRIPTION OF THE DRAWINGS

FIG. 1 shows the distribution of eye color scores determined as described in Example 1.

FIG. 2 shows the distribution of hair color scores (melanin index) determined as described in Example 2.

### DETAILED DESCRIPTION OF THE INVENTION

The present invention is based, in part, an the identification of a panel of single nucleotide polymorphisms (SNPs) that alone, or in combinations, allow an inference to be drawn as to the eye color of an individual or as to the hair color of an individual from a nucleic acid or protein sample of the individual. As disclosed herein, many of these SNPs came from a pan-genome screen and are dispersed among the chromosomes. As such the SNPs can be used individually, and in combinations, including as haploid or diploid alleles, to draw an inference regarding eye color or hair color. In addition, where the SNPs are present in the same gene or are sufficiently linked, they can be assembled into haplotypes, and haploid and/or diploid haplotype alleles can be used to infer eye color or hair color.

The term “haplotype” is used herein to refer to groupings of two or more pigmentation related (i.e., eye color related or hair color related) SNPs that are linked. As such, the SNPs can be present in the same gene or in adjacent genes or in a gene and an adjacent intergenic region, or otherwise present in the genome such that they segregate non-randomly. The term “haplotype alleles” as used herein refers to a non-random combination of nucleotide occurrences of SNPs that make up a haplotype.

The term “penetrant pigmentation-related haplotype alleles” refers to haplotype alleles whose association with eye color pigmentation or hair color pigmentation is strong enough that it can be detected using simple genetics approaches. Corresponding haplotypes of penetrant pigmentation-related haplotype alleles, are referred to herein as “penetrant pigmentation-related haplotypes.” Similarly, individual nucleotide occurrences of SNPs are referred to herein as “penetrant pigmentation-related SNP nucleotide occurrences” if the association of the nucleotide occurrence with the eye color pigmentation trait (or hair color pigmentation trait) is strong enough on its own to be detected using simple genetics approaches, or if the SNP loci for the nucleotide occurrence make up part of a penetrant haplotype. The corresponding SNP loci are referred to as penetrant pigmentation-related SNPs.

The term “latent pigmentation-related haplotype alleles” refers to haplotype alleles that, in the context of one or more penetrant haplotypes, strengthen the inference of the genetic eye color pigmentation trait and/or the genetic hair color pigmentation trait. Latent pigmentation-related haplotype alleles are typically alleles whose association with eye color (or hair color) pigmentation is not strong enough to be detected with simple genetics approaches. Latent pigmentation-related SNPs are individual SNPs that make up latent pigmentation-related haplotypes. Examples of latent pigmentation related SNPs, including latent eye color related SNPs and latent hair color related SNPs, are provided in PCT Publ. No. WO 02/097047 A2, which is incorporated herein by reference.

A sample useful for practicing a method of the invention can be any biological sample of a subject that contains nucleic acid molecules, including portions of the gene sequences to be examined, or corresponding encoded polypeptides, depending on the particular method. As such, the sample can be a cell, tissue or organ sample, or can be a sample of a biological fluid such as semen, saliva, blood, and the like. A nucleic acid sample useful for practicing a method of the invention will depend, in part, on whether the SNPs to be identified are in coding regions or in non-coding regions. Thus, where at least one of the SNPs to be identified is in a non-coding region, the nucleic acid sample generally is a deoxyribonucleic acid (DNA) sample, particularly genomic DNA or an amplification product thereof. However, where heteronuclear ribonucleic acid (RNA), which includes unspliced MRNA precursor RNA molecules, is available, a cDNA or amplification product thereof can be used. Where the each of the SNPs is present in a coding region of the pigmentation gene(s), the nucleic acid sample can be DNA or RNA, or products derived therefrom, for example, amplification products. Furthermore, while the methods of the invention generally are exemplified with respect to a nucleic acid sample, it will be recognized that particular SNP alleles can be in coding regions of a gene and can result in polypeptides containing different amino acids at the positions corresponding to the SNPs due to non-degenerate codon changes. As such, in one aspect, the methods of the invention can be practiced using a sample containing polypeptides of the subject.

Methods of the invention can be practiced with respect to human subjects and, therefore, can be particularly useful for forensic analysis. In a forensic application or a method of the invention, the human nucleic acid (or polypeptide) sample can be obtained from a crime scene, using well established sampling methods. Thus, the sample can be fluid sample or a swab sample containing nucleic acid and or polypeptide of an individual for which an inference as to eye color or hair color is to be made. For example, the sample can be a swab sample, blood stain, semen stain, hair follicle, or other biological specimen, taken from a crime scene, or can be a soil sample suspected of containing biological material of a potential crime victim or perpetrator, can be material retrieved from under the finger nails of a potential crime victim, or the like, wherein nucleic acids (or polypeptides) in the sample can be used as a basis for drawing an inference as to eye color (or hair color) according to a method of the invention.

A subject that can be examined according to a method of the invention (a test subject) can be any subject, and generally is a mammalian species. As disclosed herein, the methods are particularly applicable to drawing an inference as to eye color or natural hair color of a human subject. With respect to non-human mammalian species, the methods of the invention are valuable in providing predictions of commercially valuable eye color and/or hair color phenotypes, for example, in breeding.

The Sequence Listing containing SEQ ID NOS:1 to 48 provides the SNP position, including alternative alleles (e.g., nucleotide 426, G or C for SEQ ID NO:1), and flanking nucleotide sequences of the SNP positions, useful for inferring natural eye color (SEQ IDS NOS:1 to 10 and 26 to 48) or for inferring natural hair color (SEQ ID NOS:11 to 25). In this respect, it should be noted that the present methods are useful for inferring a natural trait, including natural eye color or natural hair color, as genetically determined and characteristic of a natural population. As such, the lack of pigmentation as occurs in oculocutaneous albinism, which is associated with a mutation and not with a naturally occurring polymorphism, is not considered to be a pigmentation related trait (eye color/shade or hair color/shade) encompassed within the present invention. The flanking sequences of the SNP positions provided in SEQ ID NOS :1 to 48 allow an identification of the precise location of the SNPs in the human genome, and can serve as target sequences useful for performing methods of the invention. In addition, the Sequence Listing provides SNP marker numbers (e.g., RS2311470, see SEQ ID NO:1), which can be used to locate the exemplified SNP in a database such as that provided by the National Institutes of Health (see world wide web (www) at “ncbi.nlm.nih.gov”; SNP database). A target polynucleotide typically includes a SNP locus and/or a segment of a corresponding gene that flanks the SNP. Either the coding strand or the complementary strand (or both) comprising the SNP positions as set forth in SEQ ID NOS:1 to 48 can be examined such that an inference as to eye color or natural hair color can be drawn. Probes and primers that selectively hybridize at or near the target polynucleotide sequence, as well as specific binding pair members that can specifically bind at or near the target polynucleotide sequence, can be designed based on the disclosed gene sequences and related information.

As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying a nucleotide occurrence of a SNP. It will be recognized that, in general, some amount of non-specific hybridization is unavoidable, but is acceptable provided that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)). Confirmation that selective hybridization is provided by particular conditions can be made using control sequences.

An example of progressively higher stringency conditions is as follows:

2×SSC/0.1% SDS at about room temperature (hybridization conditions);

0.2×SSC/0.1% SDS at about room temperature (low stringency conditions);

0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer), can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments, it can be useful to detectably label a polynucleotide or oligonucleotide. Detectable labeling of a polynucleotide or oligonucleotide is well known in the art. Particular non-limiting examples of detectable labels include chemiluminescent labels, radiolabels, enzymes, haptens, or even unique oligonucleotide sequences.

A method of the identifying an eye color related SNP or a natural hair color related SNP also can be performed using a specific binding pair member. As used herein, the term “specific binding pair member” refers to a molecule that specifically binds or selectively hybridizes to another member of a specific binding pair. Specific binding pair member include, for example, probes, primers, polynucleotides, antibodies, etc. For example, a specific binding pair member can be a primer or a probe that selectively hybridizes to a target polynucleotide that includes a SNP locus, or that hybridizes to an amplification product generated using the target polynucleotide as a template, or can be an antibody that, under the appropriate conditions, selectively binds to a polypeptide containing one, but not the other, variant encoded by a polynucleotide comprising a particular SNP.

Numerous methods are known in the art for determining the nucleotide occurrence for a particular SNP in a sample. Such methods can utilize one or more oligonucleotide probes or primers, including, for example, an amplification primer pair, that selectively hybridize to a target polynucleotide, which contains one or more pigmentation-related SNP positions. Oligonucleotide probes useful in practicing a method of the invention can include, for example, an oligonucleotide that is complementary to and spans a portion of the target polynucleotide, including the position of the SNP, wherein the presence of a specific nucleotide at the position (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe.

An oligonucleotide ligation assay also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method” (Sanger, F., et al., J. Molec. Biol. 94:441, 1975; Prober et al. Science 238:336-340, 1987) and the “chemical degradation method,” “also known as the “Maxam-Gilbert method” (Maxam et al., Proc. Natl. Acad. Sci. USA 74:560, 1977) to determine the nucleotide occurrence at the SNP loci.

Methods of the invention can identify nucleotide occurrences at SNP positions using a “microsequencing” method. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP loci are described by Boyce-Jacino et al. (U.S. Pat. No. 6,294,336, which is incorporated herein by reference).

Microsequencing methods include the Genetic Bit analysis method disclosed by Goelet et al. (PCT Publ. No. WO 92/15712, which is incorporated herein by reference). Additional, primer-guided, nucleotide incorporation procedures for assaying polymorphic sites in DNA have been described and are well known (see, e.g., Komher et al, Nucl. Acids. Res. 17:7779-7784, 1989; Sokolov, Nucl. Acids Res. 18:3671, 1990; Syvanen et al., Genomics 8:684-692, 1990; Kuppuswamy et al., Proc. Natl. Acad. Sci. USA 88:1143-1147, 1991; Prezant et al, Hum. Mutat. 1:159-164, 1992; Ugozzoli et al., GATA 9:107-112, 1992; Nyren et al., Anal. Biochem. 208:171-175, 1993; and Wallace, PCT Publ. No. WO 89/10414). These methods differ from Genetic Bit™ analysis in that they all rely on the incorporation of labeled deoriboxynucleotides to discriminate between bases at a polymorphic site. In such a format, since the signal is proportional to the number of deoriboxynucleotides incorporated, polymorphisms that occur in runs of the same nucleotide can result in signals that are proportional to the length of the run (Syvanen et al. Amer. J. Hum. Genet. 52:46-59, 1993). Alternative microsequencing methods have been provided by Mundy (U.S. Pat. No. 4,656,127) and Cohen et al (French Pat. No. 2,650,840; PCT Publ. No. WO 91/02087), describing a solution-based method for determining the identity of the nucleotide of a polymorphic site (e.g., using a primer that is complementary to allelic sequences immediately 3′-to a polymorphic site).

In response to the difficulties encountered in employing gel electrophoresis to analyze sequences, alternative methods for microsequencing have been developed. Macevicz (U.S. Pat. No. 5,002,867), for example, describes a method for determining nucleic acid sequence via hybridization with multiple mixtures of oligonucleotide probes. In accordance with such method, the sequence of a target polynucleotide is determined by permitting the target to sequentially hybridize with sets of probes having an invariant nucleotide at one position, and a variant nucleotides at other positions. The Macevicz method determines the nucleotide sequence of the target by hybridizing the target with a set of probes, and then determining the number of sites that at least one member of the set is capable of hybridizing to the target (i.e., the number of “matches”). This procedure is repeated until each member of a sets of probes has been tested. Boyce-Jacino et al. (U.S. Pat. No. 6,294,336) provide a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

In one particular commercial example of a method that can be used to identify a nucleotide occurrence of one or more SNPs, the nucleotide occurrences of pigmentation-related SNPs in a sample can be determined using the SNP-IT™ method (Orchid BioSciences, Inc.; Princeton N.J.). In general, the SNP-IT™ method is a 3-step primer extension reaction. In the first step a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect calorimetric assay, mass spectrometry, fluorescence polarization, etc. Reactions can be processed in 384 well format in an automated format using a SNPstream™ instrument (Orchid BioSciences, Inc.). Phase known data can be generated by inputting phase unknown raw data from the SNPstream™ instrument into the Stephens and Donnelly's PHASE program.

The method of identifying a nucleotide occurrence in the sample for at least one eye color related SNP or hair color related SNP, as discussed above, can further include grouping the nucleotide occurrences of the SNPs into one or more haplotype alleles indicative of eye color. For example, to infer eye color of a test subject, the identified haplotype alleles can be compared to known haplotype alleles, wherein the relationship of the known haplotype alleles to eye color is known.

Identifying eye colors corresponding to one or a combination of nucleotide occurrences of eye color related SNPs (SEQ ID NOS:1 to 10 and 26 to 48) or of hair color related SNPs (SEQ ID NOS:11 to 25), according to the present methods, can be performed by comparing the nucleotide occurrence(s) of the SNPs of the test individual with known nucleotide occurrence(s) of eye color related SNPs or hair color related SNPs of reference subjects, which have known eye colors or natural hair colors, respectively. For example, the known eye colors corresponding to one or a combination of nucleotide occurrences of eye color related SNPs can be contained in a table or other list, and the nucleotide occurrences of the test individual can be compared to the table or list visually, or can be contained database, and the comparison can be made electronically, for example, using a computer.

As disclosed herein, an inference as to eye color (or hair color) can be made by comparing the nucleotide occurrence(s) of one or more eye color (or hair color) related SNPs of a test individual with known nucleotide occurrence(s) of the same SNPs of a reference individual, for whom a genotype (i.e., nucleotide occurrence(s) of eye color or hair color related SNPs) is known and informative for (i.e., associated with) a phenotype (i.e., eye color or hair color). In one embodiment, the method comprises comparing the test subject's genotype (with respect to the nucleotide occurrence(s) of eye color (or hair color) related SNPs) with text descriptions or photographs of such reference individuals, wherein the identification of a genotype of a reference individual that matches that of the test subject allows an inference as to the eye color or hair color of the test individual (see Example 1). In one aspect, the photograph is a digital photograph, which comprises digital information that can be contained in a database that can further contain a plurality of such digital information of digital photographs, each of which is associated with a known eye color and corresponding known nucleotide occurrence(s) of eye color related SNP(s) of the reference subjects in the photographs.

A method of the invention can further include identifying a photograph of a person having an eye color or eye shade related nucleotide occurrence of a SNP corresponding to the nucleotide occurrence of the same eye color or eye shade related SNP identified in the nucleic acid sample of the test individual. Such identifying can be done by manually looking through one or more files of photographs, wherein the photographs are organized, for example, according to the nucleotide occurrences of eye color related SNPs of the person in the photograph. Identifying the photograph also can be performed by scanning a database comprising a plurality of files, each file containing digital information corresponding to a digital photograph of a person having a known eye color, and identifying at least one photograph of a person having nucleotide occurrences of SNPs indicative of eye color that correspond to the nucleotide occurrences of eye color related SNPs of the test individual.

The article of manufacture, for example, a photograph of a person having a known eye color corresponding to nucleotide occurrence(s) of eye color related SNP(s) can be a digital photograph, which comprises digital information, including for the photographic image and any other information that may be relevant or desired (e.g., the age, name, or contact information of the subject in the photograph). Such digital information of one or more digital photographs can be contained in a database thus facilitating searching of the photographs and/or known eye color (or natural hair color) and corresponding eye color (or hair color) related SNPs using electronic means. As such, the present invention further provides a plurality of the articles of manufactures, including at least two digital photographs, each of which comprises digital information. Where the digital information for one or a plurality of the articles is contained in a database, it can comprise any medium suitable for containing such a database, including, for example, computer hardware or software, a magnetic tape, or a computer disc such as floppy disc, CD, or DVD. As such, the database can be accessed through a computer, which can contain the database therein, can accept a medium containing the database, or can access the database through a wired or wireless network, e.g., an intranet or internet.

The present invention also provides kits, or components of kits, useful for inferring eye color or natural hair color according to a method of the invention. Such kits can contain, for example, a plurality (e.g., 2, 3, 4, 5, or more) of hybridizing oligonucleotides, each of which has a length of at least fifteen (e.g., 15, 16, 17, 18, 19, 20, or more) contiguous nucleotides of a polynucleotide as set forth in SEQ ID NOS:1 to 10 and 26 to 48, particularly SEQ ID NOS:1 to 7 and, optionally, SEQ ID NOS:8 to 10 and/or SEQ ID NOS:26 to 48 (or a polynucleotide complementary thereto), which are useful for inferring eye color; or as set forth in SEQ ID NOS:11 to 25 (or a polynucleotide complementary thereto), which are useful for inferring hair color. The hybridizing oligonucleotides can be probes, which hybridize to a nucleotide sequence that includes the SNP position, thus allowing the identification of one or the alternative allele (e.g., a G or a C at a position corresponding to position 426 of SEQ ID NO:1, or complement thereof); or can be primers (or primer pairs), which hybridize in sufficient proximity to the SNP position such that a primer extension (or amplification) reaction can proceed to and/or through the SNP position, thus allowing the generation of primer extension (or amplification) product containing the SNP position.

The plurality of oligonucleotides of a kit can include at least four (e.g., 4, 5, 6, 7, 8, 9, 10, 15, 20, 25, 30, or more) of the hybridizing oligonucleotide (e.g., a plurality of 32 oligonucleotides useful for sampling all of the SNPs of Table 2 and/or as set forth in SEQ ID NOS:1 to 10 and 26 to 48). In one embodiment, the hybridizing oligonucleotides include at least fifteen contiguous nucleotides of at least four polynucleotides as set forth in SEQ ID NOS:1 to 7, or polynucleotides complementary to any of SEQ ID NOS:1 to 7. In another embodiment, the hybridizing oligonucleotides are specific for at least four SNPs as set forth in SEQ ID NOS:1 to 10 and 26 to 48, including at least one SNP as set forth in SEQ ID NOS:1 to 7. In still another embodiment, the hybridizing oligonucleotides are specific for at least four SNPs as set forth in SEQ ID NOS:11 to 25. A kit of the invention also can contain at least two panels of such hybridizing oligonucleotide, including, for example, a panel comprising primers as disclosed herein and a panel comprising probes as disclosed herein, wherein the probes selectively hybridize to a product generated using the primer (e.g., a primer extension product or an amplification product).

A kit of the invention can further contain additional reagents useful for practicing a method of the invention. As such, the kit can contain one or more polynucleotides comprising an eye color related SNP and/or hair color related SNP, including, for example, a polynucleotide containing an eye color (or natural hair color) SNP for which a hybridizing oligonucleotide or pair of hybridizing oligonucleotides of the kit is designed to detect, such polynucleotide(s) being useful as controls. Further, hybridizing oligonucleotides of the kit can be detectably labeled, or the kit can contain reagents useful for detectably labeling one or more of the hybridizing oligonucleotides of the kit, including different detectable labels that can be used to differentially label the hybridizing oligonucleotides; such a kit can further include reagents for linking the label to hybridizing oligonucleotides, or for detecting the labeled oligonucleotide, or the like. A kit of the invention also can contain, for example, a polymerase, particularly where hybridizing oligonucleotides of the kit include primers or amplification primer pairs; or a ligase, where the kit contains hybridizing oligonucleotides useful for an oligonucleotide ligation assay. In addition, the kit can contain appropriate buffers, deoxyribonucleotide triphosphates, etc., depending, for example, on the particular hybridizing oligonucleotides contained in the kit and the purpose for which the kit is being provided.

The following examples are intended to illustrate but not limit the invention.

### EXAMPLE 1

### Identification of SNPs Indicative of Eye Color

This example describes the identification of SNPs useful for inferring eye color from a nucleic acid sample of an individual.

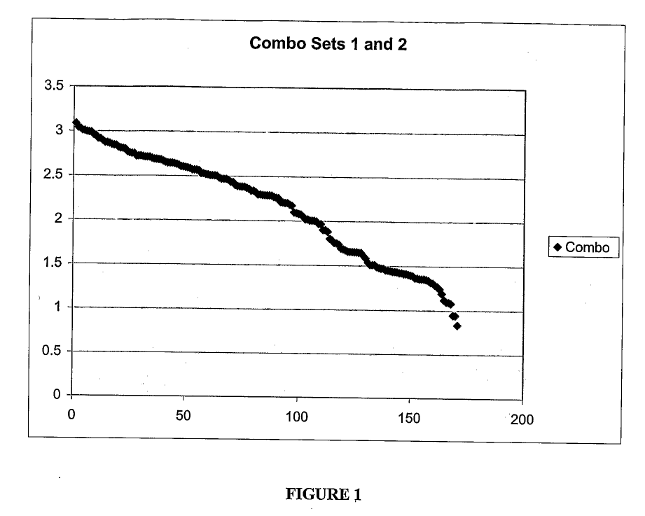

Iris colors were measured using a Cannon digital camera. Each subject peered into a cardboard box at one end, and the camera at the other end took the photo under a standardized brightness from a constant distance for each; 100 samples were collected using this method. Adobe Photoshop™ software was used to quantify the luminosity and the red/green, green/blue and red/blue wavelength reflectance ratios for the left iris; lighter eye colors had lower values for each of these variables. For each variable, the scores were scaled about the mean value. For example an eye of the average red/green value received a new scaled value of 1, with those of value below the mean converted to values less than 1 (proportional to their difference from the mean) and those greater than the mean converted to values greater than 1 (proportional to their difference from the mean). The scaled red/green, red/blue and green/blue values were summed for each eye and added together. This value was added to a scaled luminosity value for each eye to produce an eye color score for that eye. The eye color scores showed a continuous distribution (see FIG. 1).

The lightest 21 (at the top of the above distribution) were selected, and pooled into a “Light” sample; and the darkest 21 eye color samples (at the bottom of the above distribution) were selected and pooled into a “Dark” sample. A GeneChip® Mapping 10K Array and Assay Set (Affymetrix; Santa Clara Calif.) was used to screen each pool. For each of the 10,000 SNPs on the GeneChip® array, an allele frequency was calculated for the Light pool and the Dark pool. The 10,000 SNPs were ranked based on the allele frequency differential between the two groups (Delta value), a Pearson's P value statistic, and an Odds Ratio statistic on the allele frequency differential between the two groups. In addition, a screen of the pigmentation candidate genes, which included genes for which rare mutations cause catastrophic pigmentation phenotypes (e.g., albinism), was performed. SNPs in candidate genes were screened using the same sample, but genotyping individual samples rather than pools of samples. The top 100 SNPs based on the Odds Ratio statistic were selected from both approaches combined, as were all others that were in the top 100 for Delta value and Pearson's P value (even if not in the top 100 based on the Odds ratio test) to produce a set of 130 SNPs.

To validate which of the 130 SNPs were associated with iris colors, a second completely separate group of 100 samples was genotyped and ranked in the same way. The best 60 SNPs described in PCT Publ. No. WO 02/097047 A2, also were genotyped in this same sample of 100 subjects. Of the 190 candidate SNPs, approximately 30 showed either a good Delta value, Pearson's P value or Odds ratio test statistic, and 27 were used for further analysis. Table 1 shows the marker number, delta value, chromosome position, and pigmentation gene association for the SNPs of SEQ ID NOS:5, 6, and 7, which were among the 27 selected SNPs.

TABLE 1

| | | | Chromosome | |
|---|---|---|---|---|
| | Marker | DELTA | Position | GENE |
| SEQ ID NO: 5 | 1908 | 0.183333 | 15q11.2-12 | OCA2 |
| SEQ ID NO: 6 | 1916 | 0.188095 | 15q11.2-12 | OCA2 |
| SEQ ID NO: 7 | 1879 | 0.199248 | 15q11.2-12 | OCA2 |

A classification model was built using 27 SNPs identified as described above, whereby the 200 subjects used to discover them were classified into Light (green or blue eyes) or Dark (brown or hazel eyes) eye color groups. Neural nets gave a classification accuracy of about 95% within-model, and about 80% outside model. It is noted that neural nets generally require a much larger sample size for the number of variables used here. A simpler method was used to obtain a within-model accuracy of 97%.

Thirty-five SNPs, including 15 of the 27 SNPs identified as described above (and including SEQ ID NOS:5 to 7) initially were examined, and 32 SNPs were selected for further study (see Table 2). The 17 additional SNPs of the 32 were included for further study because they had interesting distributions that were helpful for classification analysis, but had less optimal P-values or delta values. In this respect, the initial 27 SNPs were selected based on a cut-off Delta value of 0.125, whereas the additional 17 SNPs selected for further study have Delta values less than 0.125.

A list of the allele frequency differential estimates from a set of about 800 self-reported eye color samples, and in a second set of 100 samples where eye color was digitally classified was prepared. Some of these SNPs were found in the first set of 800 and confirmed in the set of 100, while others were discovered from a separate set of 100 digitally qualified samples and confirmed in the set of 100. For the ones found in the first set of 800, individual genotype (not pools) data was available and, therefore, the delta values (allele frequency differential) could be compared between light and dark groups. Most of the SNPs showed similar values between the two experiments (discovery of 800 and validation of 100) but, in fact, these SNPs were originally identified in a set of 100 self-reporteds and have been validated several times in subsequent sets of 100, to get to 800 total self-reporteds, before validating them once more in the 100 digital samples (the first 800 SNPs are referred to as the discovery set, for convenience).

The delta value (allele frequency differential) was used rather than the p-value because the p-value depends on the sample size. A differential of 10% would be significant with a sample of 500 or so at the 0.05 level but not with a sample of 100. Since the interest was in confirming the original data, the p-value can be misleading because the sample sizes are unequal; the allele frequency differential is a better parameter to use. Most of the differentials were similar, showing good reproduction, even though the p-values for most of these differentials in a sample of 100 was not significant at the 0.05 level (many were close). The differences in delta value from the first 800 and the second 100 can be due to sample size effects, or because the eye colors were measured more objectively with the camera for the second 100.

Classification models incorporating the 32 SNPs (Table 2) were developed. Haplotypes were constructed based on the SNPs, and the sample genotype was compared to a database of genotypes for other samples. Those samples that matched at a combination of elements (e.g., OCA2-A+OCA2-B, OCA2-A+OCA2-C, and OCA2-B+OCA2-C; see Table 2) were retrieved, and the iris color parameters (luminosity, blue, red, green reflectance) for all samples that matched at the combinations were averaged to prove inferred iris color parameters. The database was then queried with these parameters to produce a collection of photographs of iris colors corresponding to the inferred parameters, and allowing for a visual appreciation of the inferred results (see below). Digital photographs of the irises of the individuals providing the samples were obtained, and their colors were averaged and the variance measured. The average and variance provide the parameters for the inferred iris color and its range. Using this method of inference, the iris colors of “unknown” samples, based on the genotype for these 35 SNPs, provided a blind classification accuracy of 97% when an exact genotype match existed across all of the genotypes in Table 2 in the database and 92% when only partial matches existed (e.g., only OCA2-A+OCA2-B, or OCA2-A+OCA2-B, etc.).

TABLE 2

| | | | DeCode | | |
|---|---|---|---|---|---|
| Haplotype | Gene | | Map | | |
| position | SNPID | Chromosome | Position | rs number | Sequence |
| OCA2-A-1 | 1869 | 15q11.2-q12 | 15.12 cM | rs1874835 | SEQ ID NO: 4 |
| OCA2-A-2 | 1887 | 15q11.2-q12 | 15.23 cM | rs2311470 | SEQ ID NO: 1 |
| OCA2-A-3 | 1867 | 15q11.2-q12 | 15.53 cM | rs1375170 | SEQ ID NO: 2 |
| OCA2-A-4 | 1993 | 15q11.2-q12 | 15.58 cM | rs1163825 | SEQ ID NO: 26* |
| OCA2-A-5 | 2040 | 15q11.2-q12 | 15.63 cM | rs1800411 | SEQ ID NO: 27* |
| OCA2-A-6 | 1999 | 15q11.2-q12 | 15.67 cM | rs10852218 | SEQ ID NO: 28* |
| OCA2-A-7 | 1992 | 15q11.2-q12 | 15.68 cM | rs1900758 | SEQ ID NO: 29* |
| OCA2-A-8 | 1949 | 15q11.2-q12 | 15.68 cM | rs1037208 | SEQ ID NO: 30* |
| OCA2-A-9 | 2048 | 15q11.2-q12 | 15.78 cM | rs749846 | SEQ ID NO: 31* |
| OCA2-A-10 | 1908 | 15q11.2-q12 | 16.23 cM | rs895829 | SEQ ID NO: 5 |
| OCA2-B-1 | 1916 | 15q11.2-q12 | 15.05 cM | rs1498519 | SEQ ID NO: 6 |
| OCA2-B-2 | 1905 | 15q11.2-q12 | 15.27 cM | rs1004611 | SEQ ID NO: 3 |
| OCA2-B-3 | 1873 | 15q11.2-q12 | 15.43 cM | rs3099645 | SEQ ID NO: 32 |
| OCA2-B-4 | 1870 | 15q11.2-q12 | 15.80 cM | rs3794606 | SEQ ID NO: 33 |
| OCA2-B-5 | 1895 | 15q11.2-q12 | 15.80 cM | rs2305252 | SEQ ID NO: 34 |
| OCA2-B-6 | 1879 | 15q11.2-q12 | 15.85 cM | rs895828 | SEQ ID NO: 7 |
| OCA2-C-1 | 1983 | 15q11.2-q12 | 15.05 cM | rs1800407 | SEQ ID NO: 35 |
| OCA2-C-2 | 1914 | 15q11.2-q12 | 15.15 cM | rs924314 | SEQ ID NO: 36 |
| OCA2-C-3 | 1889 | 15q11.2-q12 | 15.15 cM | rs924312 | SEQ ID NO: 37 |
| OCA2-C-4 | 1923 | 15q11.2-q12 | 15.25 cM | rs2036213 | SEQ ID NO: 38 |
| OCA2-C-5 | 1980 | 15q11.2-q12 | 15.70 cM | rs735066 | SEQ ID NO: 39 |
| OCA2-C-6 | 2043 | 15q11.2-q12 | 16.00 cM | rs1800404 | SEQ ID NO: 40 |
| TYRP1-1 | 1877 | 9p23 | 26.25 cM | rs683 | SEQ ID NO: 9* |
| TYRP1-2 | 1991 | 9p23 | 26.25 cM | rs2733832 | SEQ ID NO: 8* |
| TYRP1-3 | 2009 | 9p23 | 26.26 cM | rs2762464 | SEQ ID NO: 41* |
| ASIP-1 | 1979 | 20q11.2 | 56.943 cM | rs2424984 | SEQ ID NO: 42 |
| ASIP-2 | 1986 | 20q11.2 | 56.945 cM | rs2424987 | SEQ ID NO: 43* |
| MATP-1 | 1955 | 5p13.3 | 55.70 cM | EXON5 | SEQ ID NO: 44* |
| | | | | PHE374LEU** | |
| MATP-1 | 848 | 5p13.3 | 55.70 cM | rs35391 | SEQ ID NO: 45* |
| | 2121 | 1q22.5 | 155 cM | rs4131568 | SEQ ID NO: 46 |
| | 2193 | 4q31 | 147.6 cM | rs869537 | SEQ ID NO: 47 |
| | 2168 | 1p34 | 54.53 cM | rs1036756 | SEQ ID NO: 48 |

*see, also, Frudakis et al., Genetics 165: 2071-2083, 2003, which is incorporated herein by reference.
**not in public database.

Table 3 lists 10 SNPs, including 7 SNPs in the OCA2 gene (SEQ ID NOS:1 to 7) and 3 SNPs in the TYRP gene (SEQ ID NOS:8 to 10), that were particularly useful for inferring eye color, and indicates the eye color (shade) inference that can be drawn for a particular allele (see, also, Frudakis et al., supra, 2003). The SNP position and the alternative alleles are indicated in the Sequence Listing (SEQ ID NOS:1 to 10). Primers for detecting or identifying a SNP at a particular position can be prepared based on the disclosed sequences, or using additional flanking regions that can be identified using the exemplified sequences as probes.

TABLE 3

| | Marker | DELTA | GENE | allele/eye shade* |
|---|---|---|---|---|
| SEQ ID NO: 1 | 1887 | 0.1112573099 | OCA2 | G/lighter |
| SEQ ID NO: 2 | 1867 | 0.04047619 | OCA2 | T/darker |
| SEQ ID NO: 3 | 1905 | 0.021929825 | OCA2 | T/darker |
| SEQ ID NO: 4 | 1869 | 0.114285714 | OCA2 | T/darker |
| SEQ ID NO: 5 | 1908 | 0.183333333 | OCA2 | C/darker |
| SEQ ID NO: 6 | 1916 | 0.188095238 | OCA2 | C/darker |
| SEQ ID NO: 7 | 1879 | 0.19924812 | OCA2 | C/darker |
| SEQ ID NO: 8 | 1991 | 0.101190476 | TYRP | G/darker |
| SEQ ID NO: 9 | 1877 | 0.107142857 | TYRP | G/darker |
| SEQ ID NO: 10 | 1948 | 0078947368 | TYRP | C/darker |

*“lighter” indicates blue or green eyes; “darker” indicates brown or hazel eyes.

The iris color of a subject can be predicted from a nucleic acid sample by determining the genotype of the sample with respect to SNPs as shown in Table 2 (e.g., with one or more of the SNPs of SEQ ID NOS:1 to 7); comparing the genotype against those for known subjects in a database (i.e., subjects for whom eye color has been associated with nucleotide occurrence(s) of the SNPs; and identifying known subjects whose genotypes match the unknown sample. The iris colors of the known subjects thus provide a guide.

An inference is first made with respect to OCA2-A, OCA2-B, OCA2-C, TYRP1, ASIP and AIM haplotype phase of the SNPs of Table 2, where the SNP composition of the haplotypes is shown in Table 2 (e.g., OCA2-A comprises OCA2-A-1, OCA2-A-2, OCA2-A-3, through OCA2-A-10). The sample diploid haplotype genotype for each is one of many possible diploid haplotype genotypes that can be observed in a natural, large human population. If the haplotypes for the unknown sample are relatively common, it is likely that a reasonably sized database will contain samples of the same OCA2-A, OCA2-B, OCA2-C, TYRP1, ASIP and AIM diploid genotypes. If at least 5 of these examples exist, an average is obtained of the luminosity, red reflectance, blue reflectance and green reflectance values from the digital photographs of the irises to produce an estimate of the luminosity, red, blue and green reflectance for the unknown sample.

The average values and their standard deviations are then used as queries of the entire database, requesting all irises of luminosity, red, blue and green reflectance values that fall within the range specified by the values +/− the standard deviations. The average values and standard deviations constitute the set of estimated iris color parameters for the sample, and the collection of irises that obtains from the database query is a visual interpretation of this set of estimated iris color parameters.

If any of the haplotypes for the unknown sample are relatively uncommon, there will likely be no samples in the database of the same OCA2-A, OCA2-B, OCA2-C, TYRP1, ASIP and AIM diploid genotypes to use as a guide. In this case, the database is searched for all samples with

1) OCA2-A, OCA2-B and OCA2-C matches

2) OCA2-A, OCA2-B matches

3) OCA2-A, OCA2-C matches

4) OCA2-B, OCA2-C matches,

and an average is obtained of the luminosity, red reflectance, blue reflectance and green reflectance values from the digital photographs of the irises to produce an estimate of the luminosity, red, blue and green reflectance for the unknown sample. These average values and their standard deviations are then used as queries of the entire database, requesting all irises of luminosity, red, blue and green reflectance values that fall within the range specified by the values +/− the standard deviations. The average values and standard deviations constitute the set of estimated iris color parameters for the sample, and the collection of irises that obtains from the database query is a visual interpretation of this set of estimated iris color parameters.

This method can be modified to optimize the accuracy, by allowing for a consideration of continental and/or European ancestry when determining which samples do, or do not, “match” the unknown in the database. For example, it has been observed that, if the two OCA2-A haplotypes are both found more often in individuals of dark irises, a more accurate estimate is obtained by adding the irises for all the samples with these haplotypes in the database to the collection from which the estimated iris color parameters are determined.

Five examples of blind classifications are described as examples. CLASS1 was a sample for which the estimated iris color parameters were: Luminosity from 142.25 to 160.25, Red Reflectance from 145.7 to 169.96, Green Reflectance from 143.26 to 161.3 and Blue Reflectance from 110.39 to 145.25. Irises in the database that fall within these ranges are characteristically light in color, mostly blue, some with very small regions of brown and/or hazel and the collection of irises presented in CLASS1 constituted the visual interpretation of the estimated color parameters for this unknown sample. The actual iris color was later revealed to be of blue color.

The iris of CLASS2 was estimated to be of iris color parameters corresponding to lighter colors as well, but with a higher likelihood of brown ring around the pupil, or a brown sector upon this lighter, blue or blue/green color. The actual iris was later revealed to be a blue iris with a thin brown ring around the pupil. A similar estimate was provided for the blind sample CLASS3—blue/green with a high likelihood of a brown ring or sector upon this blue/green color. The actual iris was later revealed to fit this description accurately.

The iris of CLASS4 was estimated to be of blue/green color but with a thicker brown ring and/or larger brown sector upon this ring and the actual iris was later revealed to fit this description accurately. The iris of CLASS5 was estimated to be of darker color—from a dark green with a brown sector/ring to solid brown in color—but not blue, nor blue with brown color overlain. The actual iris fit this prediction.

When there was a match across all of the 6 haplotypes, the accuracy of this method was 97% from blind trials. When there was not such a match, the accuracy of this method was 92% from blind trials. As constituents of the OCA2-A and OCA2-B SNP groups, the SNPs shown in SEQ ID NOS:1 to 7 were particularly useful to the process of correctly inferring iris color from DNA, although restructuring the haplotype definitions to omit these SNPs still resulted in an accuracy of greater than 80%.

These results provide a panel of SNPs that can be used alone, or in combination, to draw inferences as to the eye color of an individual providing a nucleic acid sample, and demonstrate how an iris color of a subject can be predicted based on the identification of eye color related SNPs in a nucleic acid sample obtained from the subject.

### EXAMPLE 2

### Identification of SNPs Indicative of Hair Color

This Example describes the identification of SNPs that are useful for drawing an inference as to the hair color of an individual.

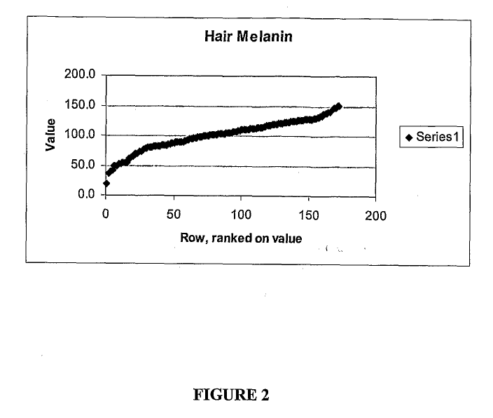

Hair color was measured using a dermaspectrometer. A reflectance reading at 650 nM is sensitive to the concentration of melanin in a sample, and is relatively insensitive to the hemoglobin concentration. Alternatively, the level of reflectance at 550 nM is due to absorbance of light by both hemoglobin and melanin. By measuring at narrow regions around these two wavelengths the melanin index (M) is computed as 100× log(1/(% reflectance at 650 nM)), and the erythema index (E) as 100× log{(% reflectance at 550 nM)/(% reflectance at 650 nM)} (Diffey et al., Brit. J Dermatol. 111:663-672, 1984, which is incorporated herein by reference). When the melanin index was calculated for 100 individuals, a continuous distribution about the mean melanin index was observed (FIG. 2).

Two pools of samples were prepared—one pool containing 21 of the lightest hair colored individuals (low melanin index), and one pool containing 21 of the darkest hair colored individuals (high melanin index). DNA was extracted from buccal swabs of the individuals and genotyped using the GeneChip® Mapping 10K Array and Assay Set (Affymetrix; see Example 1). Odds ratios, Pearson's P values and allele frequency differentials between the two groups were calculated, and about 150 of the top SNPs were selected based on these three measurements. If a SNP was in the top 130 in terms of delta value (larger is better than smaller) it was selected. In addition, if a SNP was not in the top 130 in terms of delta value, but was in the top 100 in terms of Pearson's P value (smaller is better) or Odds ratio (smaller is better), it also was selected. Sequences containing the SNPs that were particularly useful for allowing an inference to be drawn as to hair color are provided as SEQ ID NOS:11 to 25 in Sequence Listing. The SNP position and the alternative alleles are shown in the Sequence Listing for each sequence. Validation of each of the SNPs of SEQ ID NOS:11 to 25 and association with hair color can be performed as described in Example 1.

Although the invention has been described with reference to the above examples, it will be understood that modifications and variations are encompassed within the spirit and scope of the invention. Accordingly, the invention is limited only by the following claims.

What is claimed is:

1. A method for inferring natural eye color of a human subject from a nucleic acid sample of the subject, comprising identifying in the nucleic acid sample at least one nucleotide occurrence of an eye color related single nucleotide polymorphism (SNP) of an oculocutaneous albinism II (OCA2) gene, wherein the SNP comprises:
nucleotide 426 of SEQ ID NO:1, wherein a G residue indicates an increased likelihood of a lighter eye shade;
nucleotide 497 of SEQ ID NO:2, wherein a T residue indicates an increased likelihood of a darker eye shade;
nucleotide 68 of SEQ ID NO:3, wherein a T residue indicates an increased likelihood of a darker eye shade;
nucleotide 171 of SEQ ID NO:4, wherein a T residue indicates an increased likelihood of a darker eye shade;
nucleotide 533 of SEQ ID NO:5, wherein a C residue indicates an increased likelihood of a darker eye shade;
nucleotide 369 of SEQ ID NO:6, wherein a C residue indicates an increased likelihood of a darker eye shade; or
nucleotide 509 of SEQ ID NO:7, wherein a C residue indicates an increased likelihood of a darker eye shade,
wherein the lighter eye shade comprises green or blue, and wherein the darker eye shade comprises brown or hazel, thereby inferring natural eye color of the subject.

2. The method of claim 1, which comprises identifying in the nucleic acid sample nucleotide occurrences of at least two eye color related SNPs of the OCA2 gene.

3. The method of claim 1, wherein the SNP comprises an eye color related haplotype allele.

4. The method of claim 1, further comprising identifying in the nucleic acid sample at least one nucleotide occurrence of an eye color related SNP of a tyrosinase-related protein 1 (TYRP1) gene, wherein the SNP comprises:
nucleotide 172 of SEQ ID NO:8, wherein a C residue indicates an increased likelihood of a darker eye shade;
nucleotide 181 of SEQ ID NO:9, wherein a G residue indicates an increased likelihood of a darker eye shade;
nucleotide 360 of SEQ ID NO:10, wherein a C residue indicates an increased likelihood of a darker eye shade.

5. The method of claim 1, further comprising identifying in the nucleic acid sample at least one nucleotide occurrence of an eye color related SNP comprising nucleotide 21 as set forth in any of SEQ ID NOS:26 to 36 and 37 to 48, or nucleotide 26 as set forth in SEQ ID NO:37.

6. The method of claim 1, wherein identifying at least nucleotide occurrence of an one eye color related SNP of an OCA2 gene in the nucleic acid sample comprises comparing a nucleotide occurrence of the eye color related SNP of the nucleic acid sample of the subject, with known nucleotide occurrences of eye color related SNPs associated with known eye colors.

7. The method of claim 6, wherein the known nucleotide occurrences of the eye color related SNPs associated with known eye colors are contained in a database.

8. The method of claim 7, wherein the comparing is performed using a computer.

9. The method of claim 6, wherein each of the known nucleotide occurrences of the eye color related SNPs associated with a known eye color is further associated with a photograph of a person from whom a known nucleotide occurrence was determined.

10. The method of claim 9, wherein the photograph comprises a digital photograph.

11. The method of claim 10, wherein digital information comprising the digital photograph is contained in a database.

12. The method of claim 9, further comprising identifying a photograph of a person having a known nucleotide occurrence corresponding to the nucleotide occurrence of the eye color related SNP identified in the nucleic acid sample of the subject.

13. The method of claim 12, wherein identifying the photograph comprises
scanning a database comprising a plurality of files, each file comprising digital information corresponding to a digital photograph of a person having a known nucleotide occurrence of an eye color related SNP, and
identifying at least one photograph of a person having a known nucleotide occurrence of an eye color related SNP associated with a known eye color that corresponds to a nucleotide occurrence of an eye color related SNPs identified in the nucleic acid sample of the subject.

14. An article of manufacture, comprising at least one photograph of a person having a known nucleotide occurrence of an eye color related SNP associated with a known eye color.

15. The article of claim 14, which is contained in a file.

16. A plurality of files comprising the article of manufacture of claim 14, wherein files of the plurality comprise at least one photograph of a person having a known nucleotide occurrence of an eye color related SNP associated with a known eye color.

17. The file of claim 16, which comprises a plurality of photographs, wherein photographs of the plurality comprise a photograph of a person having a known nucleotide occurrence of an eye color related SNP associated with a known eye color.

18. The file of claim 17, wherein photographs of the plurality comprise photographs of different persons having the same known eye colors.

19. The article of manufacture of claim 14, wherein the at least one photograph comprises a digital photograph.

20. The article of manufacture of claim 19, wherein the digital photograph comprises digital information.

21. A kit, comprising a plurality of hybridizing oligonucleotides, which comprise at least fifteen contiguous nucleotides of at least four polynucleotides as set forth in SEQ ID NOS:1 to 7, or polynucleotides complementary thereto.

22. The kit of claim 21, wherein the hybridizing oligonucleotides comprise at least fifteen contiguous nucleotides of at least four polynucleotides as set forth in SEQ ID NOS:1 to 10 and 26 to 48, or polynucleotides complementary thereto.

23. The kit of claim 21, wherein hybridizing oligonucleotides of the plurality comprise at least one probe, at least one primer, at least one primer pair, or a combination thereof.

24. A composition for inferring natural eye color of a human subject, comprising a specific binding pair member that selectively binds to a polynucleotide comprising a nucleotide occurrence of a SNP as set forth in any of SEQ ID NOS:1 to 7, or a polypeptide encoded thereby.

25. A method for inferring natural hair color of a human subject from a nucleic acid sample of the subject, comprising identifying in the nucleic acid sample at least one nucleotide occurrence of a hair color related single nucleotide polymorphism (SNP), wherein the SNP comprises:
nucleotide 177 of SEQ ID NO:11;
nucleotide 344 of SEQ ID NO: 12;
nucleotide 24 of SEQ ID NO:13;
nucleotide 137 of SEQ ID NO:14;
nucleotide 169 of SEQ ID NO:15;
nucleotide 318 of SEQ ID NO:16;
nucleotide 122 of SEQ ID NO:17,
nucleotide 26 of SEQ ID NO:18;
nucleotide 220 of SEQ ID NO:19;
nucleotide 178 of SEQ ID NO:20;
nucleotide 26 of SEQ ID NO:21;
nucleotide 402 of SEQ ID NO:22;
nucleotide 146 of SEQ ID NO:23;
nucleotide 207 of SEQ ID NO:24; or
nucleotide 337 of SEQ ID NO:25;
wherein the nucleotide occurrence of the SNP is indicate of hair color, thereby inferring natural hair color of the subject.

26. The method of claim 25, comprising identifying at least two hair color related SNPs.

27. The method of claim 25, wherein the SNP comprises a hair color related haplotype allele.

28. A composition for inferring natural hair color of a human subject, comprising a specific binding pair member that selectively binds to a polynucleotide comprising a nucleotide occurrence of a SNP as set forth in any of SEQ ID NOS:11 to 25, or a polypeptide encoded thereby.
